SEQUENCE LISTING (91 sequences)

SEQ ID NO: 1 - RNA, 22 nt, unidentified
  source 1..22: mol_type = unassigned RNA
tggaatgtagagaagtgtgtgt

SEQ ID NO: 2 - RNA, 20 nt, unidentified
  source 1..20: mol_type = unassigned RNA
ctgggtcggtcgggctgggg

SEQ ID NO: 3 - RNA, 23 nt, unidentified
  source 1..23: mol_type = unassigned RNA
aaggattggctctaagggctggg

SEQ ID NO: 4 - RNA, 20 nt, unidentified
  source 1..20: mol_type = unassigned RNA
tgggtcggtcgggctggggc

SEQ ID NO: 5 - RNA, 21 nt, unidentified
  source 1..21: mol_type = unassigned RNA
ctgggtcggtcgggctggggc

SEQ ID NO: 6 - RNA, 22 nt, unidentified
  source 1..22: mol_type = unassigned RNA
ctgggtcggtcgggctggggcg

SEQ ID NO: 7 - RNA, 21 nt, unidentified
  source 1..21: mol_type = unassigned RNA
tgggtcggtcgggctggggcg

SEQ ID NO: 8 - RNA, 21 nt, unidentified
  source 1..21: mol_type = unassigned RNA
gctgggtcggtcgggctgggg

SEQ ID NO: 9 - RNA, 22 nt, unidentified
  source 1..22: mol_type = unassigned RNA
gctgggtcggtcgggctggggc

SEQ ID NO: 10 - RNA, 24 nt, unidentified
  source 1..24: mol_type = unassigned RNA
agggctgggtcggtcgggctgggg

SEQ ID NO: 11 - RNA, 22 nt, unidentified
  source 1..22: mol_type = unassigned RNA
ggctgggtcggtcgggctgggg

SEQ ID NO: 12 - RNA, 25 nt, unidentified
  source 1..25: mol_type = unassigned RNA
agggctgggtcggtcgggctggggc

SEQ ID NO: 13 - RNA, 23 nt, unidentified
  source 1..23: mol_type = unassigned RNA
ggctgggtcggtcgggctggggc

SEQ ID NO: 14 - RNA, 24 nt, unidentified
  source 1..24: mol_type = unassigned RNA
gggctgggtcggtcgggctggggc

SEQ ID NO: 15 - RNA, 23 nt, unidentified
  source 1..23: mol_type = unassigned RNA
gctgggtcggtcgggctggggcg

SEQ ID NO: 16 - RNA, 26 nt, unidentified
  source 1..26: mol_type = unassigned RNA
agggctgggtcggtcgggctggggcg

SEQ ID NO: 17 - RNA, 25 nt, unidentified
  source 1..25: mol_type = unassigned RNA
gggctgggtcggtcgggctggggcg

SEQ ID NO: 18 - RNA, 24 nt, unidentified
  source 1..24: mol_type = unassigned RNA
ggctgggtcggtcgggctggggcg

SEQ ID NO: 19 - RNA, 29 nt, unidentified
  source 1..29: mol_type = unassigned RNA
gcattgtggttcagtggtagaattctcgc

SEQ ID NO: 20 - RNA, 24 nt, unidentified
  source 1..24: mol_type = unassigned RNA
gcattgtggttcagtggtagaatt

SEQ ID NO: 21 - RNA, 28 nt, unidentified
  source 1..28: mol_type = unassigned RNA
gcattgtggttcagtggtagaattctcg

SEQ ID NO: 22 - RNA, 26 nt, unidentified
  source 1..26: mol_type = unassigned RNA
gcattgtggttcagtggtagaattct

SEQ ID NO: 23 - RNA, 22 nt, unidentified
  source 1..22: mol_type = unassigned RNA
agcgctgagaagacggtcgaac

SEQ ID NO: 24 - RNA, 23 nt, unidentified
  source 1..23: mol_type = unassigned RNA
agcgctgagaagacggtcgaact

SEQ ID NO: 25 - RNA, 25 nt, unidentified
  source 1..25: mol_type = unassigned RNA
agcgctgagaagacggtcgaacttg

SEQ ID NO: 26 - RNA, 26 nt, unidentified
  source 1..26: mol_type = unassigned RNA
gcgctgagaagacggtcgaacttgac

SEQ ID NO: 27 - RNA, 27 nt, unidentified
  source 1..27: mol_type = unassigned RNA
agcgctgagaagacggtcgaacttgac

SEQ ID NO: 28 - RNA, 26 nt, unidentified
  source 1..26: mol_type = unassigned RNA
agcgctgagaagacggtcgaacttga

SEQ ID NO: 29 - RNA, 21 nt, unidentified
  source 1..21: mol_type = unassigned RNA
aaaaagttaccattactgagt

SEQ ID NO: 30 - RNA, 18 nt, unidentified
  source 1..18: mol_type = unassigned RNA
tcggtcgggctggggcgc

SEQ ID NO: 31 - RNA, 27 nt, unidentified
  source 1..27: mol_type = unassigned RNA
gcgctgagaagacggtcgaacttgact

SEQ ID NO: 32 - RNA, 28 nt, unidentified
  source 1..28: mol_type = unassigned RNA
agcgctgagaagacggtcgaacttgact

SEQ ID NO: 33 - RNA, 27 nt, unidentified
  source 1..27: mol_type = unassigned RNA
agggctgggtcggtcgggctggggcgc

SEQ ID NO: 34 - RNA, 19 nt, unidentified
  source 1..19: mol_type = unassigned RNA
tcggtcgggctggggcgcg

SEQ ID NO: 35 - RNA, 23 nt, unidentified
  source 1..23: mol_type = unassigned RNA
ctgggtcggtcgggctggggcgc

SEQ ID NO: 36 - RNA, 25 nt, unidentified
  source 1..25: mol_type = unassigned RNA
ggctgggtcggtcgggctggggcgc

SEQ ID NO: 37 - RNA, 21 nt, unidentified
  source 1..21: mol_type = unassigned RNA
cggcgactctggacgcgagct

SEQ ID NO: 38 - RNA, 24 nt, unidentified
  source 1..24: mol_type = unassigned RNA
cggcggcgactctggacgcgagct

SEQ ID NO: 39 - RNA, 23 nt, unidentified
  source 1..23: mol_type = unassigned RNA
ggcggcgactctggacgcgagct

SEQ ID NO: 40 - RNA, 22 nt, unidentified
  source 1..22: mol_type = unassigned RNA
gcggcgactctggacgcgagct

SEQ ID NO: 41 - RNA, 22 nt, unidentified
  source 1..22: mol_type = unassigned RNA
tacagtactgtgataactgatt

SEQ ID NO: 42 - RNA, 28 nt, unidentified
  source 1..28: mol_type = unassigned RNA
agggctgggtcggtcgggctggggcgcg

SEQ ID NO: 43 - RNA, 24 nt, unidentified
  source 1..24: mol_type = unassigned RNA
ctgggtcggtcgggctggggcgcg

SEQ ID NO: 44 - RNA, 20 nt, unidentified
  source 1..20: mol_type = unassigned RNA
gtcggtcgggctggggcgcg

SEQ ID NO: 45 - RNA, 26 nt, unidentified
  source 1..26: mol_type = unassigned RNA
ggctgggtcggtcgggctggggcgcg

SEQ ID NO: 46 - RNA, 25 nt, unidentified
  source 1..25: mol_type = unassigned RNA
gctgggtcggtcgggctggggcgcg

SEQ ID NO: 47 - RNA, 22 nt, unidentified
  source 1..22: mol_type = unassigned RNA
tacagtactgtgataactgact

SEQ ID NO: 48 - RNA, 21 nt, unidentified
  source 1..21: mol_type = unassigned RNA
acagtactgtgataactgact

SEQ ID NO: 49 - RNA, 21 nt, unidentified
  source 1..21: mol_type = unassigned RNA
tccgactgtttaattaaaaca

SEQ ID NO: 50 - RNA, 23 nt, unidentified
  source 1..23: mol_type = unassigned RNA
tctagtccgactttgtgaaatga

SEQ ID NO: 51 - RNA, 21 nt, unidentified
  source 1..21: mol_type = unassigned RNA
atccgactgtttaattaaaac

SEQ ID NO: 52 - RNA, 22 nt, unidentified
  source 1..22: mol_type = unassigned RNA
atccgactgtttaattaaaaca

SEQ ID NO: 53 - RNA, 26 nt, unidentified
  source 1..26: mol_type = unassigned RNA
gggaatccgactgtttaattaaaaca

SEQ ID NO: 54 - RNA, 22 nt, unidentified
  source 1..22: mol_type = unassigned RNA
ccgaaagatggtgaactatgcc

SEQ ID NO: 55 - RNA, 19 nt, unidentified
  source 1..19: mol_type = unassigned RNA
tcgtaacaaggtttccgta

SEQ ID NO: 56 - RNA, 24 nt, unidentified
  source 1..24: mol_type = unassigned RNA
acccgaaagatggtgaactatgcc

SEQ ID NO: 57 - RNA, 20 nt, unidentified
  source 1..20: mol_type = unassigned RNA
tgtacacaccgcccgtcgct

SEQ ID NO: 58 - RNA, 18 nt, unidentified
  source 1..18: mol_type = unassigned RNA
taacaaggtttccgtagg

SEQ ID NO: 59 - RNA, 22 nt, unidentified
  source 1..22: mol_type = unassigned RNA
tttgtacacaccgcccgtcgct

SEQ ID NO: 60 - RNA, 18 nt, unidentified
  source 1..18: mol_type = unassigned RNA
cgggataaggattggctc

SEQ ID NO: 61 - RNA, 19 nt, unidentified
  source 1..19: mol_type = unassigned RNA
tcgggataaggattggctc

SEQ ID NO: 62 - RNA, 19 nt, unidentified
  source 1..19: mol_type = unassigned RNA
gggataaggattggctcta

SEQ ID NO: 63 - RNA, 20 nt, unidentified
  source 1..20: mol_type = unassigned RNA
ttcgggataaggattggctc

SEQ ID NO: 64 - RNA, 26 nt, unidentified
  source 1..26: mol_type = unassigned RNA
ataggggcgaaagactaatcgaacca

SEQ ID NO: 65 - RNA, 21 nt, unidentified
  source 1..21: mol_type = unassigned RNA
cgaaagatggtgaactatgct

SEQ ID NO: 66 - RNA, 23 nt, unidentified
  source 1..23: mol_type = unassigned RNA
tgtacacaccgcccgtcgctact

SEQ ID NO: 67 - RNA, 26 nt, unidentified
  source 1..26: mol_type = unassigned RNA
agggaacgtgagctgggtttagaccg

SEQ ID NO: 68 - RNA, 23 nt, unidentified
  source 1..23: mol_type = unassigned RNA
gtaacttcgggataaggattggc

SEQ ID NO: 69 - RNA, 21 nt, unidentified
  source 1..21: mol_type = unassigned RNA
cacgactctcggcaacggata

SEQ ID NO: 70 - RNA, 27 nt, unidentified
  source 1..27: mol_type = unassigned RNA
gggtctgtagctcagctggtagagcac

SEQ ID NO: 71 - RNA, 24 nt, unidentified
  source 1..24: mol_type = unassigned RNA
cgtaacttcgggataaggattggc

SEQ ID NO: 72 - RNA, 25 nt, unidentified
  source 1..25: mol_type = unassigned RNA
cgtaacttcgggataaggattggct

SEQ ID NO: 73 - RNA, 26 nt, unidentified
  source 1..26: mol_type = unassigned RNA
cgtaacttcgggataaggattggctc

SEQ ID NO: 74 - RNA, 25 nt, unidentified
  source 1..25: mol_type = unassigned RNA
ccgtaacttcgggataaggattggc

SEQ ID NO: 75 - RNA, 26 nt, unidentified
  source 1..26: mol_type = unassigned RNA
ccgtaacttcgggataaggattggct

SEQ ID NO: 76 - RNA, 27 nt, unidentified
  source 1..27: mol_type = unassigned RNA
ccgtaacttcgggataaggattggctc

SEQ ID NO: 77 - RNA, 29 nt, unidentified
  source 1..29: mol_type = unassigned RNA
gggtctgtagctcagctggttagagcacc

SEQ ID NO: 78 - RNA, 22 nt, unidentified
  source 1..22: mol_type = unassigned RNA
acaatgtaggtaagggaagtcg

SEQ ID NO: 79 - RNA, 26 nt, unidentified
  source 1..26: mol_type = unassigned RNA
agggaacgtgagctgggattagaccg

SEQ ID NO: 80 - RNA, 19 nt, unidentified
  source 1..19: mol_type = unassigned RNA
cacacgactctcggcaacg

SEQ ID NO: 81 - RNA, 24 nt, unidentified
  source 1..24: mol_type = unassigned RNA
cgcgggctctgcccgttgctctga

SEQ ID NO: 82 - RNA, 18 nt, unidentified
  source 1..18: mol_type = unassigned RNA
cgcgtcgcacggattcgt

SEQ ID NO: 83 - RNA, 19 nt, unidentified
  source 1..19: mol_type = unassigned RNA
ccgcgtcgcacggattcgt

SEQ ID NO: 84 - RNA, 23 nt, unidentified
  source 1..23: mol_type = unassigned RNA
acaatgtaggtaagggaagtcgg

SEQ ID NO: 85 - RNA, 19 nt, synthetic construct
  source 1..19: mol_type = other RNA
ttctccgaacgtgtcacgt

SEQ ID NO: 86 - RNA, 15 nt, unidentified
  source 1..15: mol_type = unassigned RNA
tcggtcgggctgggg

SEQ ID NO: 87 - RNA, 21 nt, unidentified
  source 1..21: mol_type = unassigned RNA
  modified_base 21: mod_base = cm
ctgggtcggtcgggctggggc

SEQ ID NO: 88 - RNA, 24 nt, unidentified
  source 1..24: mol_type = unassigned RNA
  modified_base 24: mod_base = um
gtattgtggttcagtggtagaatt

SEQ ID NO: 89 - RNA, 18 nt, unidentified
  source 1..18: mol_type = unassigned RNA
  modified_base 18: mod_base = cm
tcggtcgggctggggcgc

SEQ ID NO: 90 - RNA, 18 nt, unidentified
  source 1..18: mol_type = unassigned RNA
  modified_base 18: mod_base = gm
taacaaggtttccgtagg

SEQ ID NO: 91 - RNA, 19 nt, unidentified
  source 1..19: mol_type = unassigned RNA
  modified_base 19: mod_base = um
ccgcgtcgcacggattcgt

(19) United States
(12) Patent Application Publication
(10) Pub. No.: US 2026/0242799 A1
(43) Pub. Date: Aug. 20, 2026

(54) SRNA DERIVED FROM MEDICINAL PLANT AND APPLICATION THEREOF

(71) Applicant: BEIJING BAISHIHEKANG PHARMACEUTICAL TECHNOLOGY (BSJPHARMA) CO., LTD, Beijing (CN)

(72) Inventors: Qiang ZHANG, Beijing (CN); Chengyu JIANG, Beijing (CN); Heng ZHANG, Hefei City, Anhui (CN); Xinyi DU, Beijing (CN)

(21) Appl. No.: 19/498,242

(22) PCT Filed: Aug. 23, 2022
(86) PCT No.: PCT/CN2022/114330

§ 371 Date: Dec. 30, 2025

(30) Foreign Application Priority Data
Aug. 23, 2021 (CN) 202110970463.0

(51) Int. Cl. C12N 15/113; A61K 31/7105; A61K 31/712; A61K 38/46; A61P 19/08; C12N 15/88

(52) U.S. Cl. CPC C12N 15/1137; A61K 31/7105; A61K 31/712; A61K 38/465; A61P 19/08; C12N 15/88; C12Y 301/03001; C12N 2310/14; C12N 2310/321; C12N 2310/322; C12N 2310/3519; C12Y 301/03002

(57) ABSTRACT

Provided is sRNA derived from a medicinal plant, comprising the unmodified sRNA of any one of SEQ ID NO. 1 to SEQ ID NO. 84, a modified sRNA, a 5′-end and/or 3′-end truncated sRNA, an sRNA having a homology of 80% or greater, a fusion sRNA, a complementary or hybridized sRNA, a fully complementary double-stranded RNA or partially complementary double-stranded RNA, an sRNA containing a hairpin structure, or a combination thereof, which can be used to treat or prevent bone metabolic diseases.

### CROSS REFERENCE TO RELATED APPLICATIONS

This application is a U.S. National Stage Application filed under 35 U.S.C. § 371, based on International Patent Application No. PCT/CN2022/114330, filed on Aug. 23, 2022, which claims the priority to and the benefit of Chinese Patent Application No. 202110970463.0, filed on Aug. 23, 2021.

### SEQUENCE LISTING

The instant application contains a Sequence Listing which has been submitted electronically in XML file format and is hereby incorporated by reference in its entirety. Said XML copy, created on Feb. 11, 2026, is named 131546-00202_SL and is 83,094 bytes in size.

### TECHNICAL FIELD

The present invention belongs to the technical field of medicinal small nucleic acid molecules, and particularly relates to an sRNA isolated from a medicinal plant composition and applications thereof.

### BACKGROUND ART

Osteoporosis (OP) is the most common skeletal disease, characterized by low bone mass, deterioration of bone tissue microstructure, increased bone fragility, and susceptibility to fractures, representing a systemic bone disorder. In 2001, the National Institutes of Health defined it as a skeletal disorder characterized by compromised bone strength predisposing to an increased risk of fracture, indicating that reduced bone mass is a primary risk factor for osteoporotic fractures, but other risk factors also exist. OP can occur at any age but is most common in postmenopausal women and elderly men.

With the acceleration of population aging, the incidence of osteoporosis has followed cardiovascular disease and diabetes, rising to the third most common chronic disease, becoming a prevalent and frequently occurring disease worldwide. It seriously affects people's health and quality of life and imposes a significant economic burden on society. According to relevant predictions in 2015, the medical costs in China primarily for osteoporotic fractures (wrist, vertebral, and hip) in 2015, 2035, and 2050 are estimated to be as high as 72 billion, 132 billion, and 163 billion yuan, respectively. For the prevention and treatment of osteoporosis, the main current therapeutic drugs fall into three major categories: calcium supplements, vitamin D, and bone resorption inhibitors (including estrogen, selective estrogen receptor modulators, bisphosphonates, etc.). However, long-term use of these drugs can increase the incidence of gynecological cancers, cardiovascular diseases, and thrombosis, and presents numerous issues such as kidney damage, long treatment duration, high cost, and uncertain efficacy. This often forces patients to discontinue treatment due to intolerance of adverse reactions or inability to bear the high treatment costs. Therefore, finding new therapeutic measures against osteoporosis remains highly necessary.

sRNA (small RNA) refers to a class of ribonucleic acid fragments less than 200 nt in length. Most sRNAs are non-coding RNAs (ncRNAs), including: microRNA (miRNA), small nucleolar RNA (snoRNA), piwi-interacting RNA (piRNA), small nuclear RNA (U-RNA), small interfering RNA (siRNA), etc. sRNAs are widely present in animals, plants, and microorganisms and participate in regulating key biological processes such as cell proliferation, differentiation, metabolism, and death by inducing gene silencing.

Medicinal plants refer to plants that can be used in medicine for the prevention or treatment of diseases. China is one of the countries with the most abundant medicinal plant resources, the longest history of use, and the most detailed historical records. Currently known active components of medicinal plants include chemical small molecules, biological small molecules (glycosides, alkaloids, etc.), and biological macromolecules (proteins, etc.). However, a significant portion of the active components in medicinal plants remain unknown, and the known active components often cannot fully replicate the effects of the whole plant. Recent studies have found that medicinal plant-derived sRNAs, such as miR168a1 from rice, miR29112 from honeysuckle, HJT-sRNA-m73 from Rhodiola rosea, FvmiR168 from strawberry, and miR1594 from Arabidopsis thaliana, can all cross-species boundaries to regulate gene expression and physiological/pathological processes. These research findings suggest that sRNA may be a new type of active component in medicinal plants, opening up a completely new field for the study of their mechanisms.

A traditional Chinese medicine (TCM) formula composed of six herbs—Epimedium brevicornum (Yinyanghuo), Dipsacus asper (Xuduan), Salvia miltiorrhiza (Danshen), Anemarrhena asphodeloides (Zhimu), Psoralea corylifolia (Buguzhi), and Rehmannia glutinosa (Dihuang)—is used for treating osteoporosis caused by liver-kidney deficiency and blood stasis obstructing the collaterals. In the prior art, this TCM formula is mostly extracted with water or ethanol, and there has been no systematic research on the plant-derived sRNAs from it.

### SUMMARY OF THE INVENTION

The purpose of the present invention is to provide an sRNA derived from medicinal plants. Specifically, it is an sRNA isolated from a compound traditional Chinese medicine composition composed of Epimedium brevicornum, Dipsacus asper, Salvia miltiorrhiza, Anemarrhena asphodeloides, Psoralea corylifolia, and Rehmannia glutinosa.

Another purpose of the present invention is to provide uses of the above-mentioned medicinal plant-derived sRNA. Said uses include the use for preventing, treating, or ameliorating bone metabolism disorders in a subject, and the use for preparing pharmaceutical compositions.

Another purpose of the present invention is to provide a genetic engineering expression vector for preparing the above-mentioned medicinal plant-derived sRNA, and a host cell containing said expression vector.

To achieve the above purposes, the present invention adopts the following technical solutions:

In one aspect, the present invention provides an sRNA, wherein said sRNA comprises an sRNA as set forth in any one of the following sequences or a combination thereof:
SEQ ID NO. 1, SEQ ID NO. 2, SEQ ID NO. 3, SEQ ID NO. 4, SEQ ID NO. 5, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 34, SEQ ID NO. 35, SEQ ID NO. 36, SEQ ID NO. 37, SEQ ID NO. 38, SEQ ID NO. 39, SEQ ID NO. 42, SEQ ID NO. 44, SEQ ID NO. 45, SEQ ID NO. 46, SEQ ID NO. 47, SEQ ID NO. 48, SEQ ID NO. 49, SEQ ID NO. 50, SEQ ID NO. 51, SEQ ID NO. 53, SEQ ID NO. 54, SEQ ID NO. 55, SEQ ID NO. 56, SEQ ID NO. 57, SEQ ID NO. 58, SEQ ID NO. 59, SEQ ID NO. 60, SEQ ID NO. 61, SEQ ID NO. 62, SEQ ID NO. 63, SEQ ID NO. 64, SEQ ID NO. 65, SEQ ID NO. 66, SEQ ID NO. 67, SEQ ID NO. 68, SEQ ID NO. 69, SEQ ID NO. 70, SEQ ID NO. 71, SEQ ID NO. 72, SEQ ID NO. 73, SEQ ID NO. 75, SEQ ID NO. 76, SEQ ID NO. 77, SEQ ID NO. 78, SEQ ID NO. 80, SEQ ID NO. 81, SEQ ID NO. 82, SEQ ID NO. 83, SEQ ID NO. 84. In other embodiments, the sRNA possesses an RNA as set forth in any one of the sequences SEQ ID NO. 1 to SEQ ID NO. 84 or a combination thereof.

In some embodiments, the sRNA comprises an sRNA as set forth in any one of the sequences SEQ ID NO. 1 to SEQ ID NO. 84 with truncation of 1-5 or 1-3 nucleotides at 5′ end and/or 3′ end, or a combination thereof. In other embodiments, the sRNA possesses an RNA as set forth in any one of the sequences SEQ ID NO. 1 to SEQ ID NO. 84 with truncation of 1-5 or 1-3 nucleotides at 5′ end and/or 3′ end, or a combination thereof. Said truncated sRNA exhibits the same or related biological effect as the corresponding sequence as set forth SEQ ID NO. 1 to SEQ ID NO. 84.

In some embodiments, the sRNA comprises an sRNA with a identity greater than or equal to 80%, 85%, 90%, 95% to any sRNA as set forth in SEQ ID NO. 1 to SEQ ID NO. 84, or a combination thereof. In another embodiment, the sRNA possesses an sRNA with a identity greater than or equal to 80%, 85%, 90%, 95% to any sRNA as set forth in SEQ ID NO. 1 to SEQ ID NO. 84, or a combination thereof. Said homologous sRNA exhibits the same or related biological effect as the corresponding sequence as set forth SEQ ID NO. 1 to SEQ ID NO. 84.

In some embodiments, the sRNA comprises a fusion RNA of any sRNA sequence as set forth SEQ ID NO. 1 to SEQ ID NO. 84, or a combination thereof. Said fusion sRNA exhibits the same or related biological effect as the corresponding sequence as set forth SEQ ID NO. 1 to SEQ ID NO. 84.

In some embodiments, the sRNA comprises an sRNA complementary to or hybridizing with any sRNA from SEQ ID NO. 1 to SEQ ID NO. 84, or a combination thereof. Said complementary or hybridizing sRNA exhibits the same or related biological effect as the corresponding sequence from SEQ ID NO. 1 to SEQ ID NO. 84.

In some embodiments, the sRNA comprises a completely complementary double-stranded sRNA or a partially complementary double-stranded RNA comprising any sRNA of SEQ ID NO. 1 to SEQ ID NO. 84 and formed by hybridization with a nucleic acid sequence capable of hybridizing with said sRNA, or a combination thereof. In other embodiments, the sRNA possesses a completely complementary double-stranded sRNA or a partially complementary double-stranded RNA comprising any sRNA of SEQ ID NO. 1 to SEQ ID NO. 84 and formed by hybridization with a nucleic acid sequence capable of hybridizing with said sRNA, or a combination thereof. Said completely complementary or partially complementary double-stranded sRNA exhibits the same or related biological effect as the corresponding sequence as set forth in SEQ ID NO. 1 to SEQ ID NO. 84.

In some embodiments, the sRNA is any sRNA of SEQ ID NO. 1 to SEQ ID NO. 84 and contains a hairpin structure, or a combination thereof. In another embodiment, the sRNA possesses any sRNA of SEQ ID NO. 1 to SEQ ID NO. 84 and contains a hairpin structure, or a combination thereof.

In some embodiments, the sRNA is any sRNA of SEQ ID NO. 1 to SEQ ID NO. 84 and contains a 2′-fluo-modified nucleotide, a 2′-methoxyethyl-modified nucleotide, a 2′-deoxy-modified nucleotide, a 2′-amino-modified nucleotide, a 2′-O-allyl-modified nucleotide, a 2′-C-alkyl-modified nucleotide, a 2′-hydroxy-modified nucleotide, a 2′-O-alkyl-modified nucleotide, or a combination thereof; preferably a 2′-O-methyl-modified nucleotide, and more preferably the 3′ end of the RNA is a 2′-O-methyl-modified nucleotide. The sRNA using the aforementioned modified nucleotides exhibits the same or related biological effect as the corresponding sequence as set forth in SEQ ID NO. 1 to SEQ ID NO. 84.

In some embodiments, the sRNA preferably comprises an sRNA as set forth in any one of the following sequences or a combination thereof: SEQ NO. 2, SEQ NO. 5, SEQ NO. 9, SEQ NO. 11, SEQ NO. 13, SEQ NO. 14, SEQ NO. 18, SEQ NO. 20, SEQ NO. 30, SEQ NO. 34, SEQ NO. 38, SEQ NO. 39, SEQ NO. 45, SEQ NO. 46, SEQ NO. 51, SEQ NO. 58, SEQ NO. 64, SEQ NO. 67, SEQ NO. 69, SEQ NO. 70, SEQ NO. 71, SEQ NO. 73, SEQ NO. 75, SEQ NO. 76, SEQ NO. 77, SEQ NO. 78, SEQ NO. 80, SEQ NO. 81, SEQ NO. 82, SEQ NO. 83, SEQ NO. 84.

In some embodiments, the sRNA preferably comprises an sRNA as set forth in any one of the following sequences or a combination thereof: SEQ NO. 1, SEQ NO. 2, SEQ NO. 3, SEQ NO. 4, SEQ NO. 5, SEQ NO. 7, SEQ NO. 8, SEQ NO. 10, SEQ NO. 12, SEQ NO. 13, SEQ NO. 14, SEQ NO. 15, SEQ NO. 16, SEQ NO. 17, SEQ NO. 18, SEQ NO. 19, SEQ NO. 20, SEQ NO. 21, SEQ NO. 22, SEQ NO. 23, SEQ NO. 24, SEQ NO. 25, SEQ NO. 26, SEQ NO. 27, SEQ NO. 28, SEQ NO. 29, SEQ NO. 30, SEQ NO. 31, SEQ NO. 32, SEQ NO. 34, SEQ NO. 35, SEQ NO. 36, SEQ NO. 37, SEQ NO. 38, SEQ NO. 39, SEQ NO. 42, SEQ NO. 44, SEQ NO. 46, SEQ NO. 47, SEQ NO. 48, SEQ NO. 49, SEQ NO. 50, SEQ NO. 53, SEQ NO. 54, SEQ NO. 55, SEQ NO. 56, SEQ NO. 57, SEQ NO. 58, SEQ NO. 59, SEQ NO. 60, SEQ NO. 61, SEQ NO. 62, SEQ NO. 63, SEQ NO. 64, SEQ NO. 65, SEQ NO. 66, SEQ NO. 67, SEQ NO. 68, SEQ NO. 69, SEQ NO. 71, SEQ NO. 72, SEQ NO. 73, SEQ NO. 76, SEQ NO. 77, SEQ NO. 78, SEQ NO. 83.

In some embodiments, the sRNA preferably comprises an sRNA as set forth in any one of the following sequences or a combination thereof: SEQ NO. 2, SEQ NO. 5, SEQ NO. 13, SEQ NO. 14, SEQ NO. 18, SEQ NO. 20, SEQ NO. 30, SEQ NO. 34, SEQ NO. 38, SEQ NO. 39, SEQ NO. 46, SEQ NO. 58, SEQ NO. 64, SEQ NO. 67, SEQ NO. 69, SEQ NO. 71, SEQ NO. 73, SEQ NO. 76, SEQ NO. 77, SEQ NO. 78, SEQ NO. 83.

In one aspect, the present invention provides an sRNA of formula (I):

(I)
(X₁)ₙ-UCGGUCGGGCUGGGG-(X₂)ₘ

wherein,
each of X₁ is independently selected from G-, UGGG-, CUGGG-, GCUGGG-, GGCUGGG-, GGGCUGGG-, AGGGCUGGG-, or a combination thereof;
each of X₂ is independently selected from -C, -CG, -CGC, -CGCG, or a combination thereof;
m or n=0, 1, 2, or 3;
wherein the identical part sRNA sequence of UCGGUCGGGCUGGGG is SEQ ID NO. 86.

In one embodiment, said sRNA has an sRNA with identity greater than or equal to 80%, 85%, 90%, or 95% to the sRNA of formula (I) structure.

In one embodiment, said sRNA is the complementary sequence of the sRNA of formula (I) structure.

In some embodiments, the formula (I) sRNA further comprises 1-5 nucleotides, preferably 1-3 nucleotides, fused to its 5′ end and/or 3′ end.

In some embodiments, the formula (I) sRNA further comprises a completely complementary double-stranded sRNA or a partially complementary double-stranded sRNA formed by hybridization with a nucleic acid sequence capable of hybridizing with said sRNA, or a combination thereof.

In some embodiments, the formula (I) sRNA contains a hairpin structure.

In some embodiments, the formula (I) sRNA contains a 2′-fluo-modified nucleotide, a 2′-methoxyethyl-modified nucleotide, a 2′-deoxy-modified nucleotide, a 2′-amino-modified nucleotide, a 2′-O-allyl-modified nucleotide, a 2′-C-alkyl-modified nucleotide, a 2′-hydroxy-modified nucleotide, a 2′-O-alkyl-modified nucleotide, and preferably the 3′ end of the sRNA is a 2′-O-methyl-modified nucleotide.

In some embodiments, the formula (I) sRNA comprises an sRNA as set forth in SEQ NO. 2, 4-18, 30, 33-36, 42-46, or a combination thereof. In other embodiments, the formula (I) sRNA possesses an sRNA as set forth in SEQ NO. 2, 4-18, 30, 33-36, 42-46, or a combination thereof.

In one aspect, the present invention provides an sRNA possessing an sRNA that is truncated by 1-9 nucleotides at the 5′ end and/or by 1-4 nucleotides at the 3′ end of the sRNA as set forth in SEQ ID NO. 42. Specifically, the number of truncations is selected from 1, 2, 3, 4, 5, 6, 7, 8, or 9.

In another aspect, the present invention provides a genetic engineering expression vector capable of transcribing the sRNA of the present invention. The sRNA is any of the aforementioned unmodified sRNA, modified sRNA, sRNA truncated at the 5′ end and/or 3′ end, sRNA with identity greater than or equal to 80%, 85%, 90%, 95%, fusion sRNA, complementary or hybridizing sRNA, completely complementary double-stranded RNA or partially complementary double-stranded RNA, sRNA containing a hairpin structure, or the sRNA set forth in formula (I). The genetic engineering expression vector expresses the corresponding RNA in a subject, exerts the corresponding pharmacological effects, and achieves gene therapy.

In another aspect, the present invention provides a host cell comprising the genetic engineering expression vector.

In one embodiment, the genetic engineering expression vector expresses the corresponding sRNA in a subject (e.g., a patient with bone metabolism disorder or osteoporosis), thereby exerting the corresponding pharmacological effects and achieving gene therapy. In another embodiment, the genetic engineering expression vector transcribes the corresponding sRNA in vitro, which can be transcribed using host cells or a cell-free system, and after extraction (purification), administered to the subject.

In some embodiments, the pharmaceutical composition is in a dosage form suitable for administration via gavage, oral, intravenous, subcutaneous, transdermal, intramuscular, intra-arterial, intraperitoneal, intrapulmonary, intracerebrospinal, intra-articular, intrasynovial, intrathecal, intraventricular, or inhalation routes.

In some embodiments, a drug delivery complex is provided, comprising a drug delivery carrier and the sRNA, recombinant expression vector, or host cell of the present invention. In some embodiments, said drug delivery carrier is a liposome.

In another aspect, the present invention provides a use of said sRNA, its genetic engineering expression vector, its host cell, or its pharmaceutical composition in the preparation of a medicament for preventing, treating, or ameliorating a bone metabolism disorder in a subject.

In another aspect, the present invention provides a use of said sRNA, its genetic engineering expression vector, its host cell, or its pharmaceutical composition in a medicament for preventing, treating, or ameliorating a bone metabolism disorder in a subject.

In some embodiments, the bone metabolism disorder is prevented, treated, or ameliorated by increasing the expression level or activity of the alkaline phosphatase gene and/or by decreasing the expression level or activity of tartrate-resistant acid phosphatase.

In some embodiments, the bone metabolism disorder is prevented, treated, or ameliorated by promoting osteoblast differentiation and/or by inhibiting osteoclast differentiation.

In some embodiments, the sRNA that increases the expression level or activity of the alkaline phosphatase gene or promotes osteoblast differentiation is selected from: SEQ NO. 2, SEQ NO. 5, SEQ NO. 9, SEQ NO. 11, SEQ NO. 13, SEQ NO. 14, SEQ NO. 18, SEQ NO. 20, SEQ NO. 30, SEQ NO. 34, SEQ NO. 38, SEQ NO. 39, SEQ NO. 45, SEQ NO. 46, SEQ NO. 51, SEQ NO. 58, SEQ NO. 64, SEQ NO. 67, SEQ NO. 69, SEQ NO. 70, SEQ NO. 71, SEQ NO. 73, SEQ NO. 75, SEQ NO. 76, SEQ NO. 77, SEQ NO. 78, SEQ NO. 80, SEQ NO. 81, SEQ NO. 82, SEQ NO. 83, SEQ NO. 84.

In some embodiments, the sRNA that decreases the expression level or activity of tartrate-resistant acid phosphatase or inhibits osteoclast differentiation is selected from: SEQ NO. 1, SEQ NO. 2, SEQ NO. 3, SEQ NO. 4, SEQ NO. 5, SEQ NO. 7, SEQ NO. 8, SEQ NO. 10, SEQ NO. 12, SEQ NO. 13, SEQ NO. 14, SEQ NO. 15, SEQ NO. 16, SEQ NO. 17, SEQ NO. 18, SEQ NO. 19, SEQ NO. 20, SEQ NO. 21, SEQ NO. 22, SEQ NO. 23, SEQ NO. 24, SEQ NO. 25, SEQ NO. 26, SEQ NO. 27, SEQ NO. 28, SEQ NO. 29, SEQ NO. 30, SEQ NO. 31, SEQ NO. 32, SEQ NO. 34, SEQ NO. 35, SEQ NO. 36, SEQ NO. 37, SEQ NO. 38, SEQ NO. 39, SEQ NO. 42, SEQ NO. 44, SEQ NO. 46, SEQ NO. 47, SEQ NO. 48, SEQ NO. 49, SEQ NO. 50, SEQ NO. 53, SEQ NO. 54, SEQ NO. 55, SEQ NO. 56, SEQ NO. 57, SEQ NO. 58, SEQ NO. 59, SEQ NO. 60, SEQ NO. 61, SEQ NO. 62, SEQ NO. 63, SEQ NO. 64, SEQ NO. 65, SEQ NO. 66, SEQ NO. 67, SEQ NO. 68, SEQ NO. 69, SEQ NO. 71, SEQ NO. 72, SEQ NO. 73, SEQ NO. 76, SEQ NO. 77, SEQ NO. 78, SEQ NO. 83.

In some embodiments, the sRNA is preferably SEQ NO. 2, SEQ NO. 5, SEQ NO. 13, SEQ NO. 14, SEQ NO. 18, SEQ NO. 20, SEQ NO. 30, SEQ NO. 34, SEQ NO. 38, SEQ NO. 39, SEQ NO. 46, SEQ NO. 58, SEQ NO. 64, SEQ NO. 67, SEQ NO. 69, SEQ NO. 71, SEQ NO. 73, SEQ NO. 76, SEQ NO. 77, SEQ NO. 78, SEQ NO. 83. These sRNAs simultaneously possess both (1) the effect of increasing the expression level or activity of the alkaline phosphatase gene or promoting osteoblast differentiation, and (2) the effect of decreasing the expression level or activity of tartrate-resistant acid phosphatase or inhibiting osteoclast differentiation.

In some embodiments, the bone metabolism disorder is osteoporosis, including postmenopausal osteoporosis and senile osteoporosis.

In some embodiments, the “subject” is an animal, such as: a mammal, including primates (e.g., humans, non-human primates such as monkeys and chimpanzees), non-primates (e.g., cattle, pigs, camels, llamas, horses, goats, rabbits, sheep, hamsters, guinea pigs, cats, dogs, rats, mice, horses, and whales), or birds (e.g., chickens, ducks, or geese). In one embodiment, the subject is a human.

In some embodiments, single-stranded sRNA and double-stranded RNA can be synthesized using standard methods known in the art, non-limiting examples include chemical synthesis using phosphotriester, phosphite, or phosphoramidite chemistry and solid-phase techniques, such as the solid-phase phosphoramidite triester method. The synthesized oligonucleotides are isolated and purified to obtain the target product.

### Definitions

To facilitate understanding of the present invention, certain terms are first defined. Furthermore, it should be noted that whenever a parameter value or range of values is presented, it indicates that the presented value and values and ranges between said values are also part of the present invention.

The term “including” as used herein means the phrase “including (but not limited to)” and is used interchangeably therewith. The term “comprising” as used herein means the phrase “comprising (but not limited to)” and is used interchangeably therewith.

The term “or” as used herein means “and/or” and is used interchangeably therewith, unless otherwise indicated.

“G”, “C”, “A”, “T”, and “U” generally represent nucleotides containing the bases guanine, cytosine, adenine, thymidine, and uracil, respectively. However, it should be understood that the terms “ribonucleotide” or “nucleotide” also refer to modified nucleotides. One of ordinary skill in the art will appreciate that guanine, cytosine, adenine, and uracil may be replaced by other moieties without substantially altering the base-pairing properties of oligonucleotides containing such substituted nucleotides.

“sRNA” is a type of non-coding RNA molecule present in living organisms. Unless otherwise specified, “sRNA” as used herein includes unmodified sRNA, modified sRNA, sRNA truncated at the 5′ end and/or 3′ end, sRNA with identity greater than or equal to 80%, 85%, 90%, 95%, fusion sRNA, complementary or hybridizing sRNA, completely complementary double-stranded RNA or partially complementary double-stranded RNA, sRNA containing a hairpin structure, or the sRNA shown in formula (I).

Most nucleotides in each strand of a double-stranded sRNA molecule are ribonucleotides. Each strand or both strands may also include one or more non-ribonucleotides, such as deoxyribonucleotides and/or modified nucleotides. Furthermore, the sRNA used in this specification may include chemically modified ribonucleotides; the sRNA may be modified at multiple nucleotides.

The term “modified” as used herein refers to nucleotides that independently have a modified sugar moiety, modified inter-nucleotide linkage, and/or modified nucleobase. Therefore, the term “modified sRNA” includes substitutions, additions, or deletions, such as of functional groups or atoms, in the inter-nucleotide linkage, sugar moiety, or nucleobase. Modifications suitable for the formulations of the present invention include all forms disclosed herein or known in the art. For the purposes of this specification and the claims, “sRNA” includes any of these modifications for sRNA-type molecules.

The term “fusion sRNA” refers to an sRNA sequence that, after a segment of bases is fused to its 3′ end, exhibits slower degradation and increased stability; the sequence required for complementarity or complex formation with RNA remains unchanged, and its function is unchanged after fusion.

The term “alkaline phosphatase” is one of the phenotypic markers of osteoblasts, directly reflecting the activity or functional status of osteoblasts. An increase in alkaline phosphatase indicates osteogenic differentiation. The higher the alkaline phosphatase level, the more pronounced the osteogenic differentiation. Alkaline phosphatase levels can be detected using MC3T3-E1 cells (related experiments are described in L. Darryl Quarles, et al. Distinct proliferative and differentiated stages of murine MC3T3-E1 cells in culture: an in vitro model of osteoblast development. Journal of Bone and Mineral Research, 7 (6).1992.683-692).

“Tartrate-resistant acid phosphatase” can be released into the bloodstream by osteoclasts and is a blood indicator of osteoclast activity. By detecting the activity of tartrate-resistant acid phosphatase, the degree of osteoclast differentiation can be observed, thereby assessing the effect of sRNA on inhibiting osteoclast differentiation. Higher tartrate-resistant acid phosphatase activity indicates more pronounced osteoclast differentiation. Tartrate-resistant acid phosphatase levels can be detected using the RAW264.7 cell line (related experiments are described in Patricia Collin-Osdoby. et, al. (2003). Rankl-mediated osteoclast formation from murine raw 264.7 cells. Methods Mol Biol, 80, 187-202; and William J. Boyle. et, al. (2003). Osteoclast differentiation and activation. Nature, 423 (6937), 337-342).

“Same or related biological effect” refers to having the effect of preventing, treating, or ameliorating bone metabolism disorders in a subject; increasing the expression level or activity of the alkaline phosphatase gene and/or decreasing the expression level or activity of tartrate-resistant acid phosphatase; promoting osteoblast differentiation and/or inhibiting osteoclast differentiation; or preventing, treating, or ameliorating osteoporosis symptoms; or preventing, treating, or ameliorating postmenopausal osteoporosis or senile osteoporosis.

### BRIEF DESCRIPTION OF THE DRAWINGS

FIGS. 1-3 show the relative alkaline phosphatase activity of the OP1-OP84 groups.

FIGS. 4-6 show the relative tartrate-resistant acid phosphatase activity of the OP1-OP84 groups.

### DETAILED DESCRIPTION

To make the objectives, technical solutions, and advantages of the present invention clearer, the embodiments of the present invention will be further described in detail below.

Materials and reagents whose sources are not emphasized in the present invention are common materials and reagents in the field. The use of commercially sourced kits is performed according to the kit instructions. For operations where no specific method is mentioned, they are performed according to the instructions of the commercially purchased instruments or according to the conventional operational methods known to those skilled in the art.

### Example 1: Preparation of the Traditional Chinese Medicine (TCM) Composition and Individual Herb Decoctions

Each dose of the TCM composition consisted of crude herbs including Epimedium brevicornum 24-30 g, Dipsacus asper 12-15 g, Anemarrhena asphodeloides 12-15 g, Salvia miltiorrhiza 12-15 g, Psoralea corylifolia 12-15 g, and Rehmannia glutinosa 12-15 g, decocted to obtain the TCM composition (decoction). The specific decoction steps are as follows:

Took the crude herbs Epimedium brevicornum 24-30 g, Dipsacus asper 12-15 g, Anemarrhena asphodeloides 12-15 g, Salvia miltiorrhiza 12-15 g, Psoralea corylifolia 12-15 g, and Rehmannia glutinosa 12-15 g. Added 500 mL of water, soaked in cold water for 25-35 minutes. After heating to boiling, reduced to low heat for 30-35 minutes, then increased to high heat for 10-15 minutes. Uncovered and cooled to room temperature, then poured off the supernatant liquid to obtain the TCM composition.

For the individual herbs Epimedium brevicornum, Dipsacus asper, Anemarrhena asphodeloides, Salvia miltiorrhiza, Psoralea corylifolia, and Rehmannia glutinosa, independently took 100 g of each. Added 500 mL of water, soaked in cold water for 25-35 minutes. After heating to boiling, reduced to low heat for 30-35 minutes, then increased to high heat for 10-15 minutes. Uncovered and cooled to room temperature, then poured off the supernatant liquid to obtain the six individual herb decoctions, respectively.

### Example 2: Obtaining sRNA from Individual Herb Decoctions

Step 1: Extracted total RNA from the individual herb decoctions independently using a modified CTAB method. The specific steps are as follows:
(1) Took the individual herb decoctions prepared in Example 1.
(2) Added β-mercaptoethanol and preheated (65° C.) CTAB lysis buffer to the individual herb decoction, and incubated at 65° C. for 30 minutes.
(3) Centrifuged at low temperature and high speed for 8-15 minutes.
(4) Collected the supernatant, added an equal volume of isopropanol, mixed thoroughly, and let stand at −40° C. for 20 minutes.
(5) Centrifuged at low temperature and high speed for 8-15 minutes, then discarded the supernatant.
(6) Dissolved the precipitate in DEPEC-treated water, added β-mercaptoethanol and preheated (65° C.) CTAB lysis buffer, and incubated at 65° C. for 30 minutes.
(7) Added an equal volume of chloroform and let stand at 4° C. for 15 minutes.
(8) Centrifuged at low temperature and high speed for 15-20 minutes, then collected the supernatant.
(9) Added 0.6 volumes of pre-chilled (−40° C.) isopropanol to the supernatant, mixed thoroughly, and let stand at −40° C. for 30 minutes.
(10) Centrifuged at low temperature and high speed for 8-15 minutes, then discarded the supernatant.
(12) Washed with 75% ethanol, dried to obtain the total RNA precipitate from the individual herb decoction.
Step 2: Obtained sRNA sequences from the total RNA of the individual herb decoctions obtained in Step 1. The specific method is as follows:

Used Illumina's Nextseq CN500 sequencer in 1×75 bp mode to sequence the aforementioned total RNA. The method is as follows:
1) Sample quality detection: Used Agilent 2100 to test the samples. If qualified, constructed the sRNA library using the standard Illumina library construction protocol.
2) sRNA library construction: Quality checked the constructed library using Applied Biosystems' StepOnePlus QPCR. The library is considered qualified if it shows a single peak, concentration is higher than 3 nM, and volume is greater than 25 μL.
3) cDNA cluster preparation: Mixed the sample libraries according to Illumina's standard cDNA cluster generation, mixing measurement, and quantification methods to prepare cDNA clusters.
4) Data quality control: Removed low-quality sequences where the proportion of N bases >10% (or where the number of bases with quality value Q≤3 accounts for more than 50% of the entire sequence).
5) sRNA sequencing data preprocessing. Obtained sRNA from the six individual herb decoctions, merged the data to collectively form the Chinese medicine sRNA group (sRNA group 1).

### Example 3: Obtaining sRNA Sequences from Total Human Blood RNA After Medication and Analysis of Sequencing Data

Step 1: Preparation of Total Human Blood RNA from Subjects:

Two volunteers orally took the TCM formula continuously for 7 days. Each dose consisted of one portion of the TCM composition prepared in Example 1, twice daily.

Whole blood was collected from the subjects at Od (before medication), 3d, and 7d after starting medication. Total RNA was extracted using the Trizol-LS method. The extraction steps are as follows:
(1) Added Trizol LS to 250 μl of whole blood, vortexed thoroughly for lysis, add chloroform, and let stand at 4° C. for 15 minutes.
(2) Centrifuged at low temperature and high speed for 15-20 minutes, then collected the supernatant.
(3) Added 0.6 volumes of pre-chilled (−40° C.) isopropanol to the supernatant, mixed thoroughly, and let stand at −40° C. for 30 minutes.
(4) Centrifuged at low temperature and high speed for 15-20 minutes, then discarded the supernatant.
(5) Washed with 75% ethanol, dried to obtain the RNA precipitate from each sample.
   Step 2: Obtaining sRNA from the Total Human Whole Blood RNA after Consuming the TCM Composition (Decoction) in Step 1 and Performing Sequencing:

Used Illumina's Nextseq CN500 sequencer in 1×75 bp mode to sequence the aforementioned total RNA samples separately. The method is as follows:
1) Sample quality detection: Tested the samples using Agilent 2100. If qualified, constructed the sRNA library using the standard Illumina library construction protocol.
2) sRNA library construction: Quality checked the constructed library using Applied Biosystems' StepOnePlus QPCR. The library is considered qualified if it shows a single peak, concentration is higher than 3 nM, and volume is greater than 25 μL.
3) cDNA cluster preparation: Mixed the sample libraries according to Illumina's standard cDNA cluster generation, mixing measurement, and quantification methods to prepare cDNA clusters.
4) Data quality control: Removed low-quality sequences where the proportion of N bases >10% (or where the number of bases with quality value Q≤3 accounts for more than 50% of the entire sequence).
5) sRNA sequencing data preprocessing. Obtain: the sRNA group from the blood of the first volunteer at Od (sRNA group 2), the sRNA group from the blood at 3d (sRNA group 3), and the sRNA group from the blood at 7d (sRNA group 4); the sRNA group from the blood of the second volunteer at Od (sRNA group 5), the sRNA group from the blood at 3d (sRNA group 6), and the sRNA group from the blood at 7d (sRNA group 7).

### Step 3: Sequence Screening and Expression Level Calculation

Used bowtie2 for RNA sequencing data alignment. Sequences in sRNA group 3 that intersect with sequences in sRNA group 1 were classified as sRNA group 8. Calculate sRNAs in sRNA group 8 where the abundance ratio of the corresponding sRNA sequence compared to that in sRNA group 2 is greater than or equal to 1.5 (calculation method: for a specific sRNA sequence, (number of sequencing reads in sRNA group 8)/(number of sequencing reads in sRNA group 2+0.1), the same below), yielding sRNA group 9. Sequences in sRNA group 4 that intersect with sequences in sRNA group 1 were classified as sRNA group 10. Calculated sRNAs in sRNA group 10 where the abundance ratio of the corresponding sRNA sequence compared to that in sRNA group 2 is greater than or equal to 1.5, yielding sRNA group 11.

Sequences in sRNA group 6 that intersect with sequences in sRNA group 1 were classified as sRNA group 12. Calculate sRNAs in sRNA group 12 where the abundance ratio of the corresponding sRNA sequence compared to that in sRNA group 5 is greater than or equal to 1.5, yielding sRNA group 13. Sequences in sRNA group 7 that intersect with sequences in sRNA group 1 were classified as sRNA group 14. Calculated sRNAs in sRNA group 14 where the abundance ratio of the corresponding sRNA sequence compared to that in sRNA group 5 is greater than or equal to 1.5, yielding sRNA group 15.

The combined sRNA group from sRNA groups 9, 11, 13, and 15 was considered the plant-derived sRNAs enriched in the subjects' blood after medication, see Table 1.

TABLE 1
Plant-derived sRNAs enriched in subjects' blood
after medication

| No. | Code | Sequence (5′-3′) |
|---|---|---|
| SEQ ID NO. 1 | OP1 | UGGAAUGUAGAGAAGUGUGUGU |
| SEQ ID NO. 2 | OP2 | CUGGGUCGGUCGGGCUGGGG |
| SEQ ID NO. 3 | OP3 | AAGGAUUGGCUCUAAGGGCUGGG |
| SEQ ID NO. 4 | OP4 | UGGGUCGGUCGGGCUGGGGC |
| SEQ ID NO. 5 | OP5 | CUGGGUCGGUCGGGCUGGGGC |
| SEQ ID NO. 6 | OP6 | CUGGGUCGGUCGGGCUGGGGCG |
| SEQ ID NO. 7 | OP7 | UGGGUCGGUCGGGCUGGGGCG |
| SEQ ID NO. 8 | OP8 | GCUGGGUCGGUCGGGCUGGGG |
| SEQ ID NO. 9 | OP9 | GCUGGGUCGGUCGGGCUGGGGC |
| SEQ ID NO. 10 | OP10 | AGGGCUGGGUCGGUCGGGCUGGGG |
| SEQ ID NO. 11 | OP11 | GGCUGGGUCGGUCGGGCUGGGG |
| SEQ ID NO. 12 | OP12 | AGGGCUGGGUCGGUCGGGCUGGGGC |
| SEQ ID NO. 13 | OP13 | GGCUGGGUCGGUCGGGCUGGGGC |
| SEQ ID NO. 14 | OP14 | GGGCUGGGUCGGUCGGGCUGGGGC |
| SEQ ID NO. 15 | OP15 | GCUGGGUCGGUCGGGCUGGGGCG |
| SEQ ID NO. 16 | OP16 | AGGGCUGGGUCGGUCGGGCUGGGGCG |
| SEQ ID NO. 17 | OP17 | GGGCUGGGUCGGUCGGGCUGGGGCG |
| SEQ ID NO. 18 | OP18 | GGCUGGGUCGGUCGGGCUGGGGCG |
| SEQ ID NO. 19 | OP19 | GCAUUGUGGUUCAGUGGUAGAAUUCUCGC |
| SEQ ID NO. 20 | OP20 | GCAUUGUGGUUCAGUGGUAGAAUU |
| SEQ ID NO. 21 | OP21 | GCAUUGUGGUUCAGUGGUAGAAUUCUCG |
| SEQ ID NO. 22 | OP22 | GCAUUGUGGUUCAGUGGUAGAAUUCU |
| SEQ ID NO. 23 | OP23 | AGCGCUGAGAAGACGGUCGAAC |
| SEQ ID NO. 24 | OP24 | AGCGCUGAGAAGACGGUCGAACU |
| SEQ ID NO. 25 | OP25 | AGCGCUGAGAAGACGGUCGAACUUG |
| SEQ ID NO. 26 | OP26 | GCGCUGAGAAGACGGUCGAACUUGAC |
| SEQ ID NO. 27 | OP27 | AGCGCUGAGAAGACGGUCGAACUUGAC |
| SEQ ID NO. 28 | OP28 | AGCGCUGAGAAGACGGUCGAACUUGA |
| SEQ ID NO. 29 | OP29 | AAAAAGUUACCAUUACUGAGU |
| SEQ ID NO. 30 | OP30 | UCGGUCGGGCUGGGGCGC |
| SEQ ID NO. 31 | OP31 | GCGCUGAGAAGACGGUCGAACUUGACU |
| SEQ ID NO. 32 | OP32 | AGCGCUGAGAAGACGGUCGAACUUGACU |
| SEQ ID NO. 33 | OP33 | AGGGCUGGGUCGGUCGGGCUGGGGCGC |
| SEQ ID NO. 34 | OP34 | UCGGUCGGGCUGGGGCGCG |
| SEQ ID NO. 35 | OP35 | CUGGGUCGGUCGGGCUGGGGCGC |
| SEQ ID NO. 36 | OP36 | GGCUGGGUCGGUCGGGCUGGGGCGC |
| SEQ ID NO. 37 | OP37 | CGGCGACUCUGGACGCGAGCU |
| SEQ ID NO. 38 | OP38 | CGGCGGCGACUCUGGACGCGAGCU |
| SEQ ID NO. 39 | OP39 | GGCGGCGACUCUGGACGCGAGCU |
| SEQ ID NO. 40 | OP40 | GCGGCGACUCUGGACGCGAGCU |
| SEQ ID NO. 41 | OP41 | UACAGUACUGUGAUAACUGAUU |
| SEQ ID NO. 42 | OP42 | AGGGCUGGGUCGGUCGGGCUGGGGCGCG |
| SEQ ID NO. 43 | OP43 | CUGGGUCGGUCGGGCUGGGGCGCG |
| SEQ ID NO. 44 | OP44 | GUCGGUCGGGCUGGGGCGCG |
| SEQ ID NO. 45 | OP45 | GGCUGGGUCGGUCGGGCUGGGGCGCG |
| SEQ ID NO. 46 | OP46 | GCUGGGUCGGUCGGGCUGGGGCGCG |
| SEQ ID NO. 47 | OP47 | UACAGUACUGUGAUAACUGACU |
| SEQ ID NO. 48 | OP48 | ACAGUACUGUGAUAACUGACU |
| SEQ ID NO. 49 | OP49 | UCCGACUGUUUAAUUAAAACA |
| SEQ ID NO. 50 | OP50 | UCUAGUCCGACUUUGUGAAAUGA |
| SEQ ID NO. 51 | OP51 | AUCCGACUGUUUAAUUAAAAC |
| SEQ ID NO. 52 | OP52 | AUCCGACUGUUUAAUUAAAACA |
| SEQ ID NO. 53 | OP53 | GGGAAUCCGACUGUUUAAUUAAAACA |
| SEQ ID NO. 54 | OP54 | CCGAAAGAUGGUGAACUAUGCC |
| SEQ ID NO. 55 | OP55 | UCGUAACAAGGUUUCCGUA |
| SEQ ID NO. 56 | OP56 | ACCCGAAAGAUGGUGAACUAUGCC |
| SEQ ID NO. 57 | OP57 | UGUACACACCGCCCGUCGCU |
| SEQ ID NO. 58 | OP58 | UAACAAGGUUUCCGUAGG |
| SEQ ID NO. 59 | OP59 | UUUGUACACACCGCCCGUCGCU |
| SEQ ID NO. 60 | OP60 | CGGGAUAAGGAUUGGCUC |
| SEQ ID NO. 61 | OP61 | UCGGGAUAAGGAUUGGCUC |
| SEQ ID NO. 62 | OP62 | GGGAUAAGGAUUGGCUCUA |
| SEQ ID NO. 63 | OP63 | UUCGGGAUAAGGAUUGGCUC |
| SEQ ID NO. 64 | OP64 | AUAGGGGCGAAAGACUAAUCGAACCA |
| SEQ ID NO. 65 | OP65 | CGAAAGAUGGUGAACUAUGCU |
| SEQ ID NO. 66 | OP66 | UGUACACACCGCCCGUCGCUACU |
| SEQ ID NO. 67 | OP67 | AGGGAACGUGAGCUGGGUUUAGACCG |
| SEQ ID NO. 68 | OP68 | GUAACUUCGGGAUAAGGAUUGGC |
| SEQ ID NO. 69 | OP69 | CACGACUCUCGGCAACGGAUA |
| SEQ ID NO. 70 | OP70 | GGGUCUGUAGCUCAGCUGGUAGAGCAC |
| SEQ ID NO. 71 | OP71 | CGUAACUUCGGGAUAAGGAUUGGC |
| SEQ ID NO. 72 | OP72 | CGUAACUUCGGGAUAAGGAUUGGCU |
| SEQ ID NO. 73 | OP73 | CGUAACUUCGGGAUAAGGAUUGGCUC |
| SEQ ID NO. 74 | OP74 | CCGUAACUUCGGGAUAAGGAUUGGC |
| SEQ ID NO. 75 | OP75 | CCGUAACUUCGGGAUAAGGAUUGGCU |
| SEQ ID NO. 76 | OP76 | CCGUAACUUCGGGAUAAGGAUUGGCUC |
| SEQ ID NO. 77 | OP77 | GGGUCUGUAGCUCAGCUGGUUAGAGCACC |
| SEQ ID NO. 78 | OP78 | ACAAUGUAGGUAAGGGAAGUCG |
| SEQ ID NO. 79 | OP79 | AGGGAACGUGAGCUGGGAUUAGACCG |
| SEQ ID NO. 80 | OP80 | CACACGACUCUCGGCAACG |
| SEQ ID NO. 81 | OP81 | CGCGGGCUCUGCCCGUUGCUCUGA |
| SEQ ID NO. 82 | OP82 | CGCGUCGCACGGAUUCGU |
| SEQ ID NO. 83 | OP83 | CCGCGUCGCACGGAUUCGU |
| SEQ ID NO. 84 | OP84 | ACAAUGUAGGUAAGGGAAGUCGG |

### Example 4: Testing the Osteogenic-Promoting Effect of Medicinal Plant-Derived sRNA Corresponding Double-Stranded RNA in MC3T3-E1 Cell Line

In the MC3T3-E1 cell line, appropriate amounts of ascorbic acid, sodium β-glycerophosphate, and dexamethasone were added to induce osteogenic differentiation. The effect of sRNA on osteogenic differentiation was assessed by measuring alkaline phosphatase content.

When MC3T3-E1 cells reached the logarithmic growth phase, they were seeded into 12-well plates (1 mL medium/well) and incubated overnight at 37° C. Subsequent experiments were performed after cell adhesion.

Double-stranded nonsense sequence: Synthesized blunt-ended, unmodified double-stranded RNA. The specific sequence of one strand is: 5′-UUCUCCGAACGUGUCACGU (SEQ ID NO. 85).

Plant homologous double-stranded RNA: For the 84 sRNAs shown in Table 1, identical RNA and complementary RNA were synthesized separately to form blunt-ended, unmodified double-stranded RNA.

The double-stranded nonsense sequence and unmodified double-stranded RNA were synthesized by a CRO company.

Blank group: MC3T3-E1 cells were cultured in α-MEM medium without osteogenic inducers (non-induction medium 1).

Model group: MC3T3-E1 cells were cultured in α-MEM medium containing osteogenic inducers (induction medium 1).

Positive drug group: MC3T3-E1 cells were cultured in α-MEM medium containing osteogenic inducers (induction medium 1) and supplemented with lovastatin at a final concentration of 0.04 μmol/L.

Nonsense sequence group: MC3T3-E1 cells were cultured in α-MEM medium containing osteogenic inducers (induction medium 1), and the double-stranded nonsense sequence was transfected into the cells using RNAimax transfection reagent at a final concentration of 50 nM.

Nucleic acid drug groups: MC3T3-E1 cells were cultured in α-MEM medium containing osteogenic inducers (induction medium), and 84 different plant homologous double-stranded RNAs were separately transfected into the cells using RNAimax, each at a final concentration of 50 nM.

After incubating the cells with the different components for 3 days, the cells were washed with PBS, and alkaline phosphatase content was detected using an alkaline phosphatase assay kit.

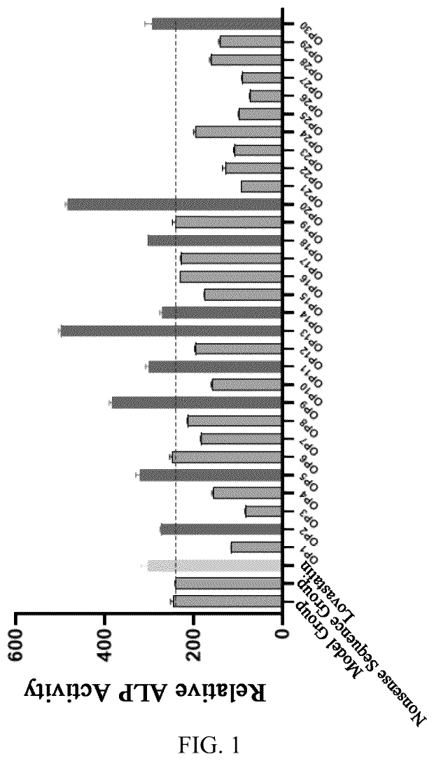

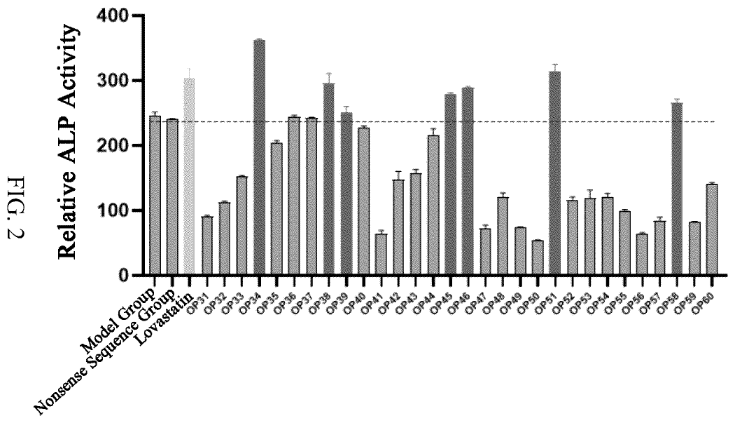

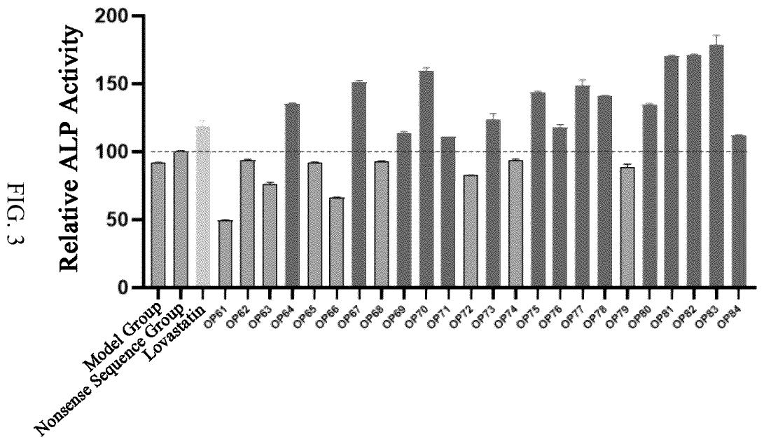

The alkaline phosphatase test results are shown in FIGS. 1-3, where ‘Con’ represents the model group, ‘NC’ represents the nonsense sequence group, ‘Lovastatin’ represents the positive drug group, and the ‘OPi’ groups indicate that one strand of the plant homologous double-stranded RNA used is identical to the sequence shown for OPi in Table 1, where ‘i’ is a natural number between 1 and 84.

The treated cells were stained for alkaline phosphatase using an alkaline phosphatase staining kit.

From the results in FIGS. 1-3, it can be seen that the alkaline phosphatase activity between the model group and the nonsense sequence group is not significantly different. However, in the nucleic acid drug groups, alkaline phosphatase activity was significantly higher after treatment with the double-stranded RNA corresponding to OP2, OP5, OP9, OP11, OP13, OP14, OP18, OP20, OP30, OP34, OP38, OP39, OP45, OP46, OP51, OP58, OP64, OP67, OP69, OP70, OP71, OP73, OP75, OP76, OP77, OP78, OP80, OP81, OP82, OP83, OP84 (31 groups) compared to the model group. Notably, OP5, OP9, OP13, OP20, OP34, OP51, OP64, OP67, OP70, OP73, OP75, OP77, OP78, OP80, OP81, OP82, OP83 showed even higher alkaline phosphatase activity than the positive drug lovastatin group.

The aforementioned 31 double-stranded sRNAs can increase the degree of osteogenic differentiation in induced MC3T3-E1 cells and can treat or alleviate osteoporosis.

### Example 5: Testing the Osteoclast Formation-Inhibiting Effect of Modified RNA Corresponding to Medicinal Plant-Derived sRNA in the RAW264.7 Cell Line

In the RAW264.7 cell line, an appropriate amount of RANKL was added to induce osteoclast differentiation, and the effect of sRNA on osteoclast differentiation was assessed.

When RAW264.7 cells reached the logarithmic growth phase, they were seeded into 12-well plates (1 mL medium/well) and incubated overnight at 37° C. Subsequent experiments were performed after cell adhesion.

Blank group: RAW264.7 cells were cultured in DMEM (low glucose) medium without osteoclast inducers (non-induction medium 2).

Model group: RAW264.7 cells were cultured in DMEM (low glucose) medium containing osteoclast inducers (induction medium 2).

Nonsense sequence group: RAW264.7 cells were cultured in DMEM (low glucose) medium containing osteoclast inducers (induction medium 2), and the aforementioned double-stranded nonsense sequence was transfected into the cells using RNAimax transfection reagent at a final concentration of 50 nM.

Nucleic acid drug groups: RAW264.7 cells were cultured in DMEM (low glucose) medium containing osteoclast inducers (induction medium 2), and the aforementioned 84 plant homologous double-stranded RNAs were separately transfected into the cells using RNAimax, each at a final concentration of 50 nM.

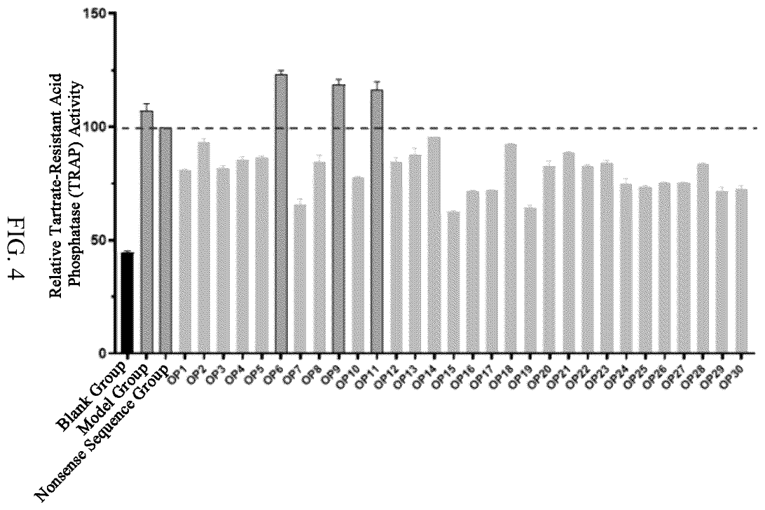

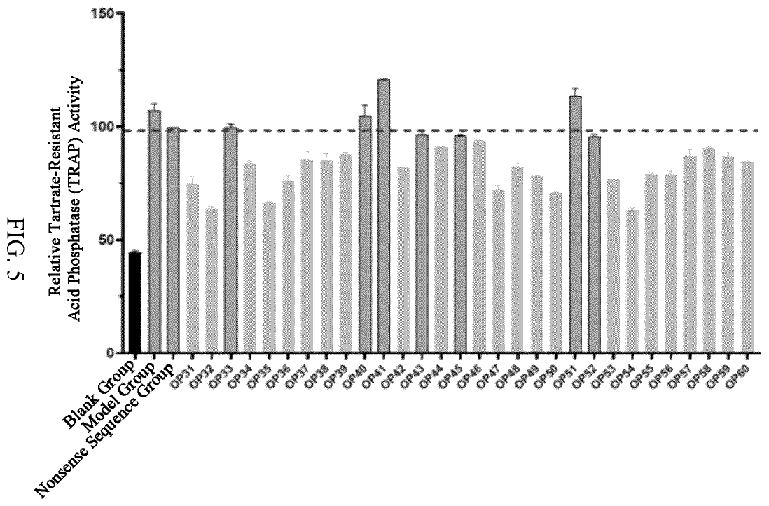

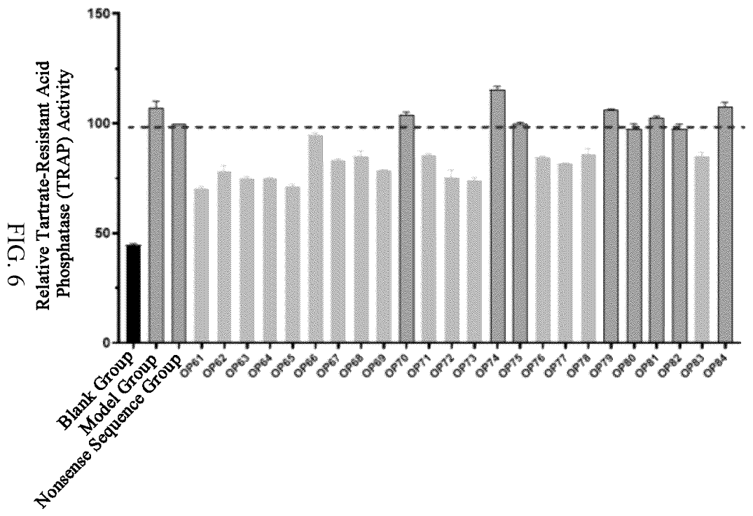

After incubating the different cell groups with the various components for 4 days, the cells were washed with PBS. Tartrate-resistant acid phosphatase content was detected using a tartrate-resistant acid phosphatase assay kit. The results are shown in FIGS. 4-6, where ‘Blank’ represents the blank group, ‘Con’ represents the model group, ‘NC’ represents the nonsense sequence group, and the ‘OPi’ groups indicate that one strand of the plant homologous double-stranded RNA used is identical to the sequence shown for OPi in Table 1, where ‘i’ is a natural number between 1 and 84.

From the results in FIGS. 4-6, it can be seen that the relative tartrate-resistant acid phosphatase activity of the model group (Con) and the nonsense sequence group (NC) is significantly greater than that of the blank group (Blank), indicating that the cell osteoclast model constructed in the present invention is effective, and the cells in the model effectively differentiate towards the osteoclast lineage compared to the blank.

Thus, it can be seen that after treatment with the nucleic acid drug groups corresponding to OP1, OP2, OP3, OP4, OP5, OP7, OP8, OP10, OP12, OP13, OP14, OP15, OP16, OP17, OP18, OP19, OP20, OP21, OP22, OP23, OP24, OP25, OP26, OP27, OP28, OP29, OP30, OP31, OP32, OP34, OP35, OP36, OP37, OP38, OP39, OP42, OP44, OP46, OP47, OP48, OP49, OP50, OP53, OP54, OP55, OP56, OP57, OP58, OP59, OP60, OP61, OP62, OP63, OP64, OP65, OP66, OP67, OP68, OP69, OP71, OP72, OP73, OP76, OP77, OP78, OP83 (a total of 66 groups), the relative tartrate-resistant acid phosphatase activity was significantly lower than that of the model group and the nonsense sequence group. These treatments effectively inhibited cell differentiation towards the osteoclast lineage and can treat or alleviate osteoporosis.

Combined with FIGS. 1-3, among the aforementioned 66 groups, the nucleic acid drug groups for OP2, OP5, OP13, OP14, OP18, OP20, OP30, OP34, OP38, OP39, OP46, OP58, OP64, OP67, OP69, OP71, OP73, OP76, OP77, OP78, OP83 (a total of 21 groups), in addition to being able to reduce tartrate-resistant acid phosphatase activity, were also able to increase alkaline phosphatase activity.

### Example 6: Effect of Modified Medicinal Plant-Derived Single-Stranded sRNA in Osteoporosis Model Mice

### 1. Establishment of Bilateral Ovariectomy Osteoporosis Mouse Model

The experiment used 6-8 week-old, 16-20 g C57BL/6 female mice. Mice underwent ovariectomy via a dorsal incision. Bilateral ovariectomized mice simulate postmenopausal osteoporosis and serve as the mouse osteoporosis model of the present invention for detecting the impact of drug intervention on osteoporotic mice.

### 2. Preparation of Experimental Drugs

### Preparation of Nonsense Sequence Control Drug:

Artificially synthesized sRNA with the specific sequence: 5′-UUCUCCGAACGUGUCACGU (SEQ ID NO. 85). The 3′-end ribonucleotide was modified with 2′-O-methylation. This modified RNA is more stable and degrades more slowly.

### Preparation of Nucleic Acid Drugs:

Artificially synthesized sRNAs were synthesized according to the sequences shown in Table 1 for OP5, OP20, OP30, OP58, and OP83. The 3′-end ribonucleotide of each was modified with 2′-O-methylation.

The 2′-O-methylated sRNAs were synthesized artificially by a CRO company.

20 nmol of the 2′-O-methylated OP5, OP20, OP30, OP58, OP83, or the nonsense sequence control drug were each dissolved in DEPC water and then mixed with lipids to form the nonsense sequence NC liposome complex, the 2′-O-methylated nucleic acid drug OP5 liposome complex, OP20 liposome complex, OP30 liposome complex, OP58 liposome complex, and OP83 liposome complex, respectively.

The contents of Xianling Gubao capsules were taken and dissolved in distilled water to form a 20 mg/mL drug suspension for use as the control group.

### 3. Experimental Grouping

Sham Operation Group (Sham group): For comparison with the model in step 1, only the adipose tissue surrounding the ovaries was removed, the ovaries were preserved, the fallopian tubes were not ligated, and suturing was performed. Distilled water was administered by gavage once daily for 5 weeks. This sham-operated mouse models trauma to assess the impact of surgical procedures and stress on experimental results and does not cause osteoporosis in mice.

Model Group (OVX group): For the model mice from step 1, distilled water was administered by gavage once daily for 5 weeks.

Nonsense Sequence Group (NC group): For the model mice from step 1, the nonsense sequence liposome complex was administered by gavage at a dose of 10 nmol per mouse, once daily for 5 weeks.

Nucleic Acid Drug Groups: For the model mice from step 1, the 2′-O-methylated nucleic drug liposome complexes for OP5 (SEQ ID NO. 87), OP20 (SEQ ID NO. 88), OP30 (SEQ ID NO. 89), OP58 (SEQ ID NO. 90), and OP83 (SEQ ID NO. 91) were administered by gavage separately. The nucleic acid drug dose was 10 nmol per mouse, once daily for 5 weeks.

Traditional Chinese Medicine Composition Group (Xianling Gubao Capsule group): For the model mice from step 1, the aforementioned drug suspension was administered by gavage. The dose of Xianling Gubao capsule contents was 236 mg/kg of mouse body weight, once daily for 5 weeks.

All the aforementioned groups began drug administration 12 days after surgery and were housed under identical environmental conditions.

### 4. MicroCT Experiment

Mice were euthanized by cervical dislocation after blood collection via eyeball enucleation. Femurs were isolated, connective and muscle tissue on the femur surface were removed, and the femurs were fixed in 4% paraformaldehyde aqueous solution for 24-48 hours. After washing with PBS three times, they were stored in 75% ethanol. MicroCT scanning was performed using a microCT instrument (manufacturer: SCANCO Medical AG, model: uCT-100).

Longitudinal images of the distal femur, transverse images of the distal femur, and transverse images of the distal femur from another angle were obtained.

CT images showed that the bone density of the model group was significantly lower than that of the sham group. The reduced bone density in the model group could be improved by the traditional Chinese medicine composition group, indicating that the osteoporosis induced by this model can be alleviated by appropriate drugs, making this model suitable for screening anti-osteoporosis drugs.

The osteoporotic condition of the nonsense sequence group was comparable to that of the model group. The bone density of the traditional Chinese medicine composition group and the nucleic acid drug groups was essentially equivalent to or slightly higher than that of the sham group. The bone density of the traditional Chinese medicine composition group and the nucleic acid drug groups was significantly higher than that of the model group and the nonsense sequence group. The nucleic acid drugs tested in this example can effectively treat or alleviate osteoporosis.

The accompanying software system of the instrument was used for the following statistical analysis of the Micro CT results:
(1) BMD (Bone Mineral Density): Results showed that the bone density of the tested nucleic acid drug groups was slightly higher than that of the sham group, equivalent to 1.12-1.34 times that of the model group, and 1.11-1.33 times that of the nonsense sequence group.
(2) Tb.Th (Trabecular Thickness): Results showed that the trabecular thickness of the tested nucleic acid drug groups was higher than that of the sham group, equivalent to 1.39-1.68 times that of the model group, and 1.42-1.70 times that of the nonsense sequence group.
(3) Ct.Th (Cortical Thickness): Results showed that the cortical thickness of the tested nucleic acid drug groups was close to that of the sham group, equivalent to 1.18-1.42 times that of the model group, and 1.18-1.42 times that of the nonsense sequence group.

The data for the tested nucleic acid drug groups were superior to those of the Xianling Gubao capsule group, indicating their potential use in treating or alleviating osteoporosis. The model group and the nonsense sequence group were very close in all parameters, indicating that in this test, the nonsense sequence had no significant effect on the various indicators, and the observed changes were caused by the traditional Chinese medicine and the nucleic acid drug liposome complexes.

It is understood by those skilled in the art that the present invention can be implemented through other embodiments that do not depart from its spirit or essential characteristics. Therefore, the disclosed embodiments are illustrative in all respects and not exclusive. All changes within the scope of the present invention or equivalent thereto are encompassed by the present invention.

1. An sRNA, comprising an sRNA as set forth in any one of the sequences SEQ ID NO. 1 to SEQ ID NO. 84 or a combination thereof.

2. The sRNA according to claim 1, wherein the sRNA is selected from the group consisting of:
an sRNA comprising an sRNA as set forth in any one of the sequences SEQ ID NO. 1 to SEQ ID NO. 84 with truncation of 1-5 or 1-3 nucleotides at 5′ end and/or 3′ end, or a combination thereof; or
an sRNA comprising an sRNA having an identity of greater than or equal to 80%, 85%, 90%, or 95% with the sRNA as set forth in any one of the sequences SEQ ID NO. 1 to SEQ ID NO. 84, or a combination thereof; or
a fusion sRNA comprising the sequence of any sRNA of SEQ ID NO. 1 to SEQ ID NO. 84, or a combination thereof; or
an sRNA comprising an sRNA complementary to or hybridizing with any sRNA of SEQ ID NO. 1 to SEQ ID NO. 84 RNA, or a combination thereof; or
a completely complementary double-stranded sRNA or a partially complementary double-stranded sRNA comprising any sRNA of SEQ ID NO. 1 to SEQ ID NO. 84 and formed by hybridization with a nucleic acid sequence capable of hybridizing with said sRNA, or a combination thereof; or
an sRNA comprising any sRNA of SEQ ID NO. 1 to SEQ ID NO. 84 and containing a hairpin structure, or a combination thereof; or
any sRNA of SEQ ID NO. 1 to SEQ ID NO. 84 containing a 2′-fluo-modified nucleotide, a 2′-methoxyethyl-modified nucleotide, a 2′-deoxy-modified nucleotide, a 2′-amino-modified nucleotide, a 2′-O-allyl-modified nucleotide, a 2′-C-alkyl-modified nucleotide, a 2′-hydroxy-modified nucleotide, a 2′-O-alkyl-modified nucleotide, preferably a 2′-O-methyl-modified nucleotide, preferably the 3′ end of the sRNA is a 2′-O-methyl-modified nucleotide.

3. The sRNA according to claim 1, wherein the sRNA comprises an sRNA as set forth in any one of the following sequences or a combination thereof:
SEQ NO. 2, SEQ NO. 5, SEQ NO. 9, SEQ NO. 11, SEQ NO. 13, SEQ NO. 14, SEQ NO. 18, SEQ NO. 20, SEQ NO. 30, SEQ NO. 34, SEQ NO. 38, SEQ NO. 39, SEQ NO. 45, SEQ NO. 46, SEQ NO. 51, SEQ NO. 58, SEQ NO. 64, SEQ NO. 67, SEQ NO. 69, SEQ NO. 70, SEQ NO. 71, SEQ NO. 73, SEQ NO. 75, SEQ NO. 76, SEQ NO. 77, SEQ NO. 78, SEQ NO. 80, SEQ NO. 81, SEQ NO. 82, SEQ NO. 83, SEQ NO. 84.

4. The sRNA according to claim 1- or 2, wherein the sRNA comprises an sRNA as set forth in any one of the following sequences or a combination thereof:
SEQ NO. 1, SEQ NO. 2, SEQ NO. 3, SEQ NO. 4, SEQ NO. 5, SEQ NO. 7, SEQ NO. 8, SEQ NO. 10, SEQ NO. 12, SEQ NO. 13, SEQ NO. 14, SEQ NO. 15, SEQ NO. 16, SEQ NO. 17, SEQ NO. 18, SEQ NO. 19, SEQ NO. 20, SEQ NO. 21, SEQ NO. 22, SEQ NO. 23, SEQ NO. 24, SEQ NO. 25, SEQ NO. 26, SEQ NO. 27, SEQ NO. 28, SEQ NO. 29, SEQ NO. 30, SEQ NO. 31, SEQ NO. 32, SEQ NO. 34, SEQ NO. 35, SEQ NO. 36, SEQ NO. 37, SEQ NO. 38, SEQ NO. 39, SEQ NO. 42, SEQ NO. 44, SEQ NO. 46, SEQ NO. 47, SEQ NO. 48, SEQ NO. 49, SEQ NO. 50, SEQ NO. 53, SEQ NO. 54, SEQ NO. 55, SEQ NO. 56, SEQ NO. 57, SEQ NO. 58, SEQ NO. 59, SEQ NO. 60, SEQ NO. 61, SEQ NO. 62, SEQ NO. 63, SEQ NO. 64, SEQ NO. 65, SEQ NO. 66, SEQ NO. 67, SEQ NO. 68, SEQ NO. 69, SEQ NO. 71, SEQ NO. 72, SEQ NO. 73, SEQ NO. 76, SEQ NO. 77, SEQ NO. 78, SEQ NO. 83.

5. The sRNA according to claim 1, wherein the sRNA comprises an sRNA as set forth in any one of the following sequences or a combination thereof:
SEQ NO. 2, SEQ NO. 5, SEQ NO. 13, SEQ NO. 14, SEQ NO. 18, SEQ NO. 20, SEQ NO. 30, SEQ NO. 34, SEQ NO. 38, SEQ NO. 39, SEQ NO. 46, SEQ NO. 58, SEQ NO. 64, SEQ NO. 67, SEQ NO. 69, SEQ NO. 71, SEQ NO. 73, SEQ NO. 76, SEQ NO. 77, SEQ NO. 78, SEQ NO. 83.

6. A genetically engineered expression vector, wherein the genetically engineered expression vector is capable of transcribing the sRNA according to claim 1.

7. A host cell, wherein the host cell comprises the recombinant expression vector according to claim 6.

8. A pharmaceutical composition, comprising the sRNA according to claim 1, and a pharmaceutically acceptable excipient,
wherein the pharmaceutical composition is in a dosage form suitable for administration via gavage, oral, intravenous, subcutaneous, transdermal, intramuscular, intra-arterial, intraperitoneal, intrapulmonary, intracerebrospinal, intra-articular, intrasynovial, intrathecal, intraventricular, or inhalation routes.

9. The composition according to claim 8, wherein the composition is in a form of liposome.

10. A method of preventing, treating, or ameliorating a bone metabolism disorder in a subject, comprising administering the sRNA according to claim 1 to the subject.

11. The method according to claim 10, wherein the bone metabolism disorder is prevented, treated, or ameliorated by increasing the expression level or activity of alkaline phosphatase gene and/or decreasing the expression level or activity of tartrate-resistant acid phosphatase.

12. The method according to claim 10, wherein the bone metabolism disorder is prevented, treated, or ameliorated by promoting osteoblast differentiation and/or inhibiting osteoclast differentiation.

13. The method according to claim 11, wherein the sRNA that increases the expression level or activity of the alkaline phosphatase gene or promotes osteoblast differentiation is selected from:
SEQ NO. 2, SEQ NO. 5, SEQ NO. 9, SEQ NO. 11, SEQ NO. 13, SEQ NO. 14, SEQ NO. 18, SEQ NO. 20, SEQ NO. 30, SEQ NO. 34, SEQ NO. 38, SEQ NO. 39, SEQ NO. 45, SEQ NO. 46, SEQ NO. 51, SEQ NO. 58, SEQ NO. 64, SEQ NO. 67, SEQ NO. 69, SEQ NO. 70, SEQ NO. 71, SEQ NO. 73, SEQ NO. 75, SEQ NO. 76, SEQ NO. 77, SEQ NO. 78, SEQ NO. 80, SEQ NO. 81, SEQ NO. 82, SEQ NO. 83, SEQ NO. 84.

14. The method according to claim 11, wherein the sRNA that decreases the expression level or activity of tartrate-resistant acid phosphatase or inhibits osteoclast differentiation is selected from:
SEQ NO. 1, SEQ NO. 2, SEQ NO. 3, SEQ NO. 4, SEQ NO. 5, SEQ NO. 7, SEQ NO. 8, SEQ NO. 10, SEQ NO. 12, SEQ NO. 13, SEQ NO. 14, SEQ NO. 15, SEQ NO. 16, SEQ NO. 17, SEQ NO. 18, SEQ NO. 19, SEQ NO. 20, SEQ NO. 21, SEQ NO. 22, SEQ NO. 23, SEQ NO. 24, SEQ NO. 25, SEQ NO. 26, SEQ NO. 27, SEQ NO. 28, SEQ NO. 29, SEQ NO. 30, SEQ NO. 31, SEQ NO. 32, SEQ NO. 34, SEQ NO. 35, SEQ NO. 36, SEQ NO. 37, SEQ NO. 38, SEQ NO. 39, SEQ NO. 42, SEQ NO. 44, SEQ NO. 46, SEQ NO. 47, SEQ NO. 48, SEQ NO. 49, SEQ NO. 50, SEQ NO. 53, SEQ NO. 54, SEQ NO. 55, SEQ NO. 56, SEQ NO. 57, SEQ NO. 58, SEQ NO. 59, SEQ NO. 60, SEQ NO. 61, SEQ NO. 62, SEQ NO. 63, SEQ NO. 64, SEQ NO. 65, SEQ NO. 66, SEQ NO. 67, SEQ NO. 68, SEQ NO. 69, SEQ NO. 71, SEQ NO. 72, SEQ NO. 73, SEQ NO. 76, SEQ NO. 77, SEQ NO. 78, SEQ NO. 83.

15. The method according to claim 11, wherein the sRNA is selected from:
SEQ NO. 2, SEQ NO. 5, SEQ NO. 13, SEQ NO. 14, SEQ NO. 18, SEQ NO. 20, SEQ NO. 30, SEQ NO. 34, SEQ NO. 38, SEQ NO. 39, SEQ NO. 46, SEQ NO. 58, SEQ NO. 64, SEQ NO. 67, SEQ NO. 69, SEQ NO. 71, SEQ NO. 73, SEQ NO. 76, SEQ NO. 77, SEQ NO. 78, SEQ NO. 83.

16. The method according to claim 10, wherein the bone metabolism disorder is osteoporosis, preferably postmenopausal osteoporosis or senile osteoporosis.

17. A method of preventing, treating, or ameliorating a bone metabolism disorder in a subject, comprising administering the pharmaceutical composition according to claim 8 to the subject.

18-23. (canceled)

24. An sRNA of formula (I),
(I)
(X₁)ₙ-UCGGUCGGGCUGGGG-(X₂)ₘ
wherein,
each of X₁ is independently selected from G-, UGGG-, CUGGG-, GCUGGG-, GGCUGGG-, GGGCUGGG-, AGGGCUGGG-, or a combination thereof;
each of X₂ is independently selected from -C, -CG, -CGC, -CGCG, or a combination thereof;
m or n=0, 1, 2, or 3.
